(19) United States
(12) Patent Application Publication
(10) Pub. No.: US 2011/0077471 A1
(43) Pub. Date: Mar. 31, 2011

(54) TREATMENT AND PREVENTION OF OVERWEIGHT AND OBESITY BY ALTERING VISUAL PERCEPTION OF FOOD DURING CONSUMPTION

(76) Inventor: Brian Spencer King, (US)

(21) Appl. No.: 12/728,769

(22) Filed: Mar. 22, 2010

Related U.S. Application Data
(60) Provisional application No. 61/162,204, filed on Mar. 20, 2009.

(51) Int. Cl. A61B 5/00

(52) U.S. Cl. 600/300

(57) ABSTRACT

Provided are methods for reducing food consumption, and methods for altering behavioral, neurological, psychological, or physiological responses to a food portion, mainly for use by an obese or overweight user, or by a user at risk of weight gain. Also provided are user-interactive systems and apparatuses for employing such methods.

### REFERENCE TO RELATED APPLICATIONS

This application claims an invention which was disclosed in Provisional Application No. 61162204 filed Mar. 20, 2009, entitled “TREATMENT AND PREVENTION OF OVERWEIGHT AND OBESITY BY ALTERING VISUAL PERPECPTION OF FOOD DURING CONSUMPTION.”. The benefit under 35 USC §119(e) of the United States provisional application is hereby claimed, and the aforementioned application is hereby incorporated by reference.

### BACKGROUND

1. Technical Field

The present invention relates to methods and apparatuses for treating or reducing obesity and related conditions by altering the visual perception of food during consumption, such as by altering the perception of the image of the food the user of the invention grasps at immediately prior to consumption or by altering the users perception of the ratio between food size and the size of a reference object immediately prior to consumption.

2. Description of the Related Art

Obesity is a condition in which excess body fat has accumulated to an extent that health may be negatively affected. Obesity is commonly defined as a body mass index (BMI) of 30 kg/m2 or higher. This measurement distinguishes obesity from pre-obesity (i.e., ‘overweight’), which is typically defined as a BMI of 25 kg/m2 but less than 30 kg/m2.

Excessive body weight is associated with various diseases, including, for example, cardiovascular diseases, diabetes mellitus type 2, obstructive sleep apnea, certain types of cancer, and osteoarthritis, among others. In this regard, obesity has been found to reduce life expectancy. Most cases of obesity are believed to result from a combination of excessive caloric intake, lack of physical activity, and genetic susceptibility, with a limited number of cases attributed solely to genetics, medical reasons, and/or psychiatric illness.

Many patients who lose weight eventually regain the lost weight. This pattern of regaining lost weight is known as weight cycling or yo-yo dieting. Weight cycling presents additional health risks beyond those present in cases of overweight and obesity. Many patients who regain weight regain more weight than they originally lost.

The primary treatment for overweight and obesity is dieting and physical exercise. If these treatments fail, treatments may rely on anti-obesity drugs and (in severe cases of obesity) bariatric surgery. There is no treatment or program such that primary care providers can reliably induce and maintain that weight loss in patients.

Obesity is one of the leading preventable causes of death worldwide.

Certain authorities view obesity as one of the most serious public health problem of the 21st century. Therefore, it is desirable to identify new methods for treating and/or preventing obesity.

### BRIEF SUMMARY

Certain embodiments of the present invention relate generally to methods for reducing food consumption by an overweight or obese user when employing a user-interactive system while eating, relative to food consumption when the user-interactive system is not employed, and without having to determine food portion size, the method comprising:
(a) providing a user-interactive system that comprises (i) a camera means for generating an image of a bite of food that is to be consumed by the user, (ii) an image-processing means for receiving from said camera means the image of the bite of food that is to be consumed by the user and magnifying the image to obtain a magnified image of the bite of food, (iii) a display means for receiving from the image-processing means and displaying the magnified image of the bite of food to obtain a displayed magnified image of the bite of food, (iv) a food source, and (v) a reference object;
(b) allowing the user to present to the camera means a bite of food that is to be ingested, under conditions that permit the user-interactive system to generate and magnify an image of the bite of food and display a magnified image of the bite of food on the display means, said bite of food comprising all or a portion of the food source;
(c) allowing the user to visually compare the reference object to the displayed magnified image of the bite of food for a time period immediately before ingestion of the bite of food;
(d) allowing the user to ingest the bite of food; and
(e) permitting the user to repeat steps (b)-(d) zero, one, or a plurality of times up to a satiety point, and thereby reducing a first level of food consumption by the user when employing the user-interactive system while eating, relative to a second level of food consumption by the user when the user-interactive system is not employed.

Certain embodiments relate to methods for altering behavioral, neurological, psychological, or physiological response to a food portion by an overweight or obese user or by a user at risk of weight gain, when employing a user-interactive system while eating, relative to behavioral, neurological, psychological, or physiological response to a food portion when the user-interactive system is not employed, and without having to determine food portion size, the method comprising:
(a) providing a user-interactive system that comprises (i) a camera means for generating an image of a bite of food that is to be consumed by the user, (ii) an image-processing means for receiving from said camera means the image of the bite of food that is to be consumed by the user and magnifying the image to obtain a magnified image of the bite of food, (iii) a display means for receiving from the image-processing means and displaying the magnified image of the bite of food to obtain a displayed magnified image of the bite of food, (iv) a food source, and (v) a reference object;
(b) allowing the user to present to the camera means a bite of food that is to be ingested, under conditions that permit the user-interactive system to generate and magnify an image of the bite of food and display a magnified image of the bite of food on the display means, said bite of food comprising all or a portion of the food source;
(c) allowing the user to visually compare the reference object to the displayed magnified image of the bite of food for a time period immediately before ingestion of the bite of food;
(d) allowing the user to ingest the bite of food; and
(e) permitting the user to repeat steps (b)-(d) zero, one, or a plurality of times up to a satiety point, and thereby altering a first level of behavioral, neurological, psychological, or physiological response to a food portion by the user when employing the user-interactive system while eating, relative to a second level of behavioral, neurological, psychological, or physiological response to the food portion by the user when the user-interactive system is not employed.

In certain embodiments, the reference object is a hand of the user. In certain embodiments, the reference object is selected from the group consisting of an artificial hand, a scale model of an artificial hand, an image of a hand and an image of a scale model of a hand. In certain embodiments, the artificial hand is an artificial human hand and the image of a hand is an image of a human hand. In certain embodiments, the reference object is an article of manufacture having dimensions that conform to a manufacturing industry standard. In certain embodiments, the article of manufacture is selected from the group consisting of:
(a) an electronic or electrical device or a component thereof that is selected from the group consisting of: a removable battery for a portable electronic device, an alternating current (AC) wall plug, an AC wall outlet, a light bulb, a compact disc, a telephone handset, a computer mouse,
(b) a unit of currency that is selected from a paper bill of standard denomination and a metal coin of standard denomination,
(c) a common household item that is selected from a twelve-ounce beverage can, a twelve-ounce beverage bottle, a playing card, a deck of playing cards, a pair of eyeglasses, a pair of sunglasses, a ruler,
(d) a scale model of any article of manufacture according to (a)-(c).

In certain embodiments, the user is selected from an overweight user, an obese user, a user having one or more genetic, familial or environmental risk factors for an obesity-linked cancer, a user having one or more genetic, familial or environmental risk factors for stroke, a user having a history of yo-yo dieting or weight cycling, and a user having a condition that is selected from Type 1 diabetes, Type 2 diabetes, impaired glucose tolerance, insulin resistance, sleep apnea, osteoarthritis, gout, high blood pressure, and abnormal blood lipid levels.

In certain embodiments, the user has (i) leptin signaling deficiency, (ii) decreased left dorsolateral prefrontal cortex (LDLPFC) activity in response to a meal, or (iii) atypical hedonic response to a food portion. In certain embodiments, leptin signaling, activity in the LDLPFC, or the hedonic response to a meal is modulated by increasing a degree of magnification of the magnified image of food which is compared to the reference object.

In certain embodiments, the camera means is selected from the group consisting of a closed-circuit television camera, a digital camera, a digital video camera, a web camera and a projection camera. In certain embodiments, the camera means is attached to the display means by a support member. In certain embodiments, the image-processing means is attached to at least one of the camera means and the display means.

In certain embodiments, the image is selected from the group consisting of an optically projected image, a television image and a digital image. In certain embodiments, the user-interactive system comprises a computer operatively coupled to the camera means, the image-processing means and the display means, said computer being programmed to control one or more of image generation, image magnification and image display.

In certain embodiments, the display means is selected from a computer screen, a flat-screen television, a cathode ray tube (CRT) display and a light-projection screen. In certain embodiments, the user-interactive system comprises a lighting means for illuminating the bite of food when it is presented to the camera means.

In certain embodiments, such as in step (b) of the above methods, the user presents the bite of food to the camera using a first hand of the user or a utensil held in the first hand of the user, and wherein in step (c) the reference object that is visually compared is a second hand of the user, said second hand being contralateral to said first hand. In certain embodiments, the visual comparison between the second hand and the image of the bite of food is facilitated by the second hand executing a complete, partial, feigned, or imagined grasping movement directed at the enlarged image of the bite of food prior to consumption.

In certain embodiments, the image-processing means comprises a computer processor and a computer-readable medium having executable instructions stored thereon for converting an image signal generated by the camera means into a display signal that can be displayed by the display means. In certain embodiments, the user-interactive system comprises a protective tray that protects one or more of the camera means, the image-processing means, the display means or any other element of the device from spillage.

In certain embodiments, the display means comprises a head-mounted visual display apparatus, and wherein in step (c) of the methods above, the user is allowed (i) to visually compare the reference object to one or a plurality of displayed magnified, minified, or both magnified and minified images of the bite of food, said plurality comprising images having different magnification factors, or (ii) to visually compare one or a plurality of magnified, minified or both magnified and minified images of the reference object to one or a plurality of displayed magnified, minified, or both magnified and minified images of the bite of food, said plurality of images of the bite of food and said plurality of images of the reference object each comprising images having different magnification factors.

### BRIEF DESCRIPTION OF THE SEVERAL VIEWS OF THE DRAWINGS

FIG. 1 shows a preferred embodiment of the invention comprising a thin display (7) placed behind a plate (9). A camera (5) and light source (4) extend from the display via a retractable arm (6). The user (1) brings a utensil with a bite of food (3) (in this case, held by the right hand (2)) in position relative to the camera, which produces a magnified image on the display (8). From this position, the user initiates a comparison between the reference object (in this case, the unmagnified contralateral left hand (10)) and the magnified image (8) by grasping at the magnified food image with left hand. After initiating this comparison, the user brings the food to their mouth with the right hand. The user then may repeat this process for each bite of food, thereby altering either portion size consumed or the behavioral, neurological, psychological and/or physiological response to the portion consumed.

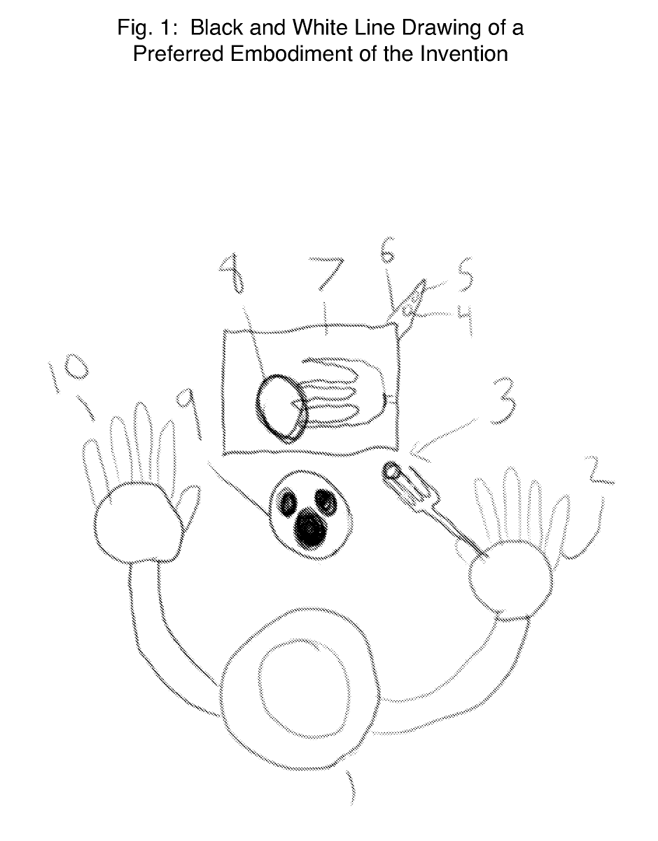

### DETAILED DESCRIPTION

Embodiments of the present invention relate generally to methods and apparatuses for reducing food consumption, and/or methods for altering behavioral, neurological, psychological, or physiological responses to a food portion, typically by an overweight or obese user, or by a user at risk of weight gain. These methods are based, in part and in certain embodiments, on the surprising discovery that increasing the user's perception of the relative ratio of a food portion size to the user's body size will alter the conscious experience of multimodal sensory experiences while eating. According to non-limiting theory, the neural correlates of these alterations to conscious experience will cause the brain signal to the body that a point of satiety or fullness has been reached with less food consumed than would be the case without using the invention, and will thereby reduce food consumption by the user accordingly. As such, the methods described herein may be practiced independent of the typical dieting strategies, which rely heavily on pre-planning the number and/or size of meals in a given day to limit caloric intake over a period of time. In particular, and in contrast to previously described approaches, according to these and related embodiments, the user need not determine food portion size in order to practice the present methods or to realize their benefits. Further, the methods described herein may be combined synergistically with many diet and weight loss strategies.

As one general example, this result may be accomplished by methods that provide a magnified image image of a bite of food, which, prior to its consumption, can then be compared to a normal or a smaller-than-normal sized reference object, such as the user's hand that is not being used to eat the food, or a scale model of a hand. User-interface systems and apparatuses are described herein to allow the practice of these and related methods.

Without being bound by any one theory, it is believed that food-based marketing and advertising have created unrealistic expectation about food, including enhanced and unrealistic perceptions about food size (e.g., food portion size, food “bite” size, etc.) and the ratio between food size and body size, thereby corrupting expectations about food size and resulting in learning about food from mental images of food portions that are widely distorted compared to the mental images of food portions and ratios between food portions and body size while eating healthy portions of food. For instance, food is often packaged with images that are “enhanced to show texture,” causing them to appear 2, 3, 5, and sometimes 20 or more times as large as the food portions actually are. These unrealistic images are often made to appear larger still by surrounding the magnified food images with cartoons with unrealistically small hands or optical illusions such as the drotse effect. Further, television viewing has been shown to be an independent factor for overweight and obesity.

In this light, it is believed further according to non-limiting theory that certain eating-related regulatory deficiencies (e.g., leptin biological signaling deficiencies) and behavioral abnormalities (e.g., overeating) may result from visual cues in the environment during the process by which an individual learns about food, which learning-associated cues do not match the visual cues that are perceived while actually eating real food, including unconsciously learned expectations as to the ratio of food size to the user's body size. Thus, by practicing the instant methods, which alter the user's perception of ratio of food size to body size, users may have the same visual experience while eating food that they were taught to expect from marketing and packaging materials.

Given the relative simplicity and non-invasiveness of the instant methods and apparatuses, which rely in part on many basic tools or devices already utilized in every day life (e.g., cameras, video screens, displays or monitors, etc.), the methods, user interfaces, and apparatuses of the present invention provide an affordable and practical means for treating or reducing obesity, and/or treating or reducing any of the diseases or conditions associated with obesity, especially as compared to the presently available pharmaceutical and surgical methods. The contemplated embodiments are not, however, intended to be so limited and may also find beneficial uses in other contexts for altering (e.g., increasing or decreasing in a statistically significant manner) a behavioral, neurological, psychological and/or physiological response to food, including the contexts of overweight/obesity and/or diabetes in certain embodiments and also including the contexts of conditions in which an increase in food consumption may be desired, such as anorexia, failure-to-thrive/wasting disorders, and the like, or appetite loss associated with cancer and other therapies such as chemotherapy and radiation therapy, and/or accidental radiation exposure.

Unless defined otherwise, all technical and scientific terms used herein have the same meaning as commonly understood by those of ordinary skill in the art to which the present embodiments relate. Although any methods and materials similar or equivalent to those described herein can be used in the practice or testing of the present embodiments, certain preferred methods and materials are described herein. For the purposes of the present invention, the following terms are defined below. All references referred to herein, including all cited patent publications and publications from non-patent literature, are incorporated by reference in their entirety.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 25, 10, 9, 8, 7, 6, 5, 4, 3, 2 or 1% relative to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not significantly interfere with or contribute to the activity or action specified in the disclosure for the listed elements. Thus, the phrase “consisting essentially of” indicates that the listed elements are required or mandatory, but that other elements are optional and may or may not be present depending upon whether or not they significantly affect (e.g., increase or decrease in a statistically significant manner) the activity or action of the listed elements.

A user may in certain preferred embodiments include an overweight user, an obese user, a user having one or more genetic, familial or environmental risk factors for an obesity-linked cancer, a user having one or more genetic, familial or environmental risk factors for stroke, a user having a history of yo-yo dieting or weight cycling, and a user having a condition that is selected from Type 1 diabetes, Type 2 diabetes, impaired glucose tolerance, insulin resistance, sleep apnea, osteoarthritis, gout, high blood pressure, and abnormal blood lipid levels. As discussed elsewhere herein, other types of users are contemplated, such as any human subject for whom altering (e.g., increasing or decreasing in a statistically significant manner) food consumption, or altering a behavioral, neurological, psychological and/or physiological response to food, may be beneficial. Users who are primarily interested in applying these means towards alteration of the conscious experience of eating, without regard to health effects, are also contemplated, such as chefs, restaurant patrons, culinary students, etc.

An overweight or obese user relates generally to subject having a body mass index (BMI) of 20, 21, 22, 23, 24, 25, 26, 27, 28, 29, 30 kg/m2 or higher, but can also include those subjects having a lower BMI or other measurement of obesity, especially if the subjects are at risk of weight gain. An overweight or obese user may also be identified based on a calculation of body fat percentage, in which total body fat is expressed as a percentage of total body weight. It is generally agreed that men with more than 25% body fat and women with more than 33% body fat are obese. Also, the absolute waist circumference (typically, a measurement greater that 102 cm in men and greater than 88 cm in women indicates obesity) and/or the waist-hip ratio (i.e., the circumference of the waist divided by that of the hips, obesity being generally indicated by a value greater than 0.9 for men and a value greater than 0.85 for women) may both be seen as measurements of obesity.

Factors that may be utilized to identify a user at risk for weight gain are known in the art, and may include, for example, genetic factors, diet, lifestyle, previous weight loss, current weight, body type, BMI, body fat percentage, time spent viewing television, etc. Users at risk for weight gain may include users who have previously lost weight or that are already considered obese or overweight, according to the measurements described herein and/or known in the art (e.g., BMI, etc.), and may include those users having measurements below the defined thresholds for obesity.

A user may also have a leptin signaling deficiency, decreased brain left dorsolateral prefrontal cortex (LDLPFC) activity in response to a meal, or a hedonic response to a food portion.

Leptin is a 16 kDa protein hormone that plays a key role in regulating energy intake and energy expenditure, including appetite and metabolism. Leptin is one of the most important adipose derived hormones and is produced almost exclusively by adipocytes. Leptin binds to the ventromedial nucleus of the hypothalamus, typically referred to as the appetite center. Leptin signals to the brain that the body has had enough to eat, i.e., that the body has reached satiety. In the absence of leptin, or in the case of reduced levels of leptin, the brain is not capable of adequately detecting satiety, and the subject continues to eat additional food even though unnecessary.

Thus, certain subjects may have a leptin signaling deficiency due to homozygous mutations (e.g., Ob/Ob; same allelic form of gene locus on both members of the pair of chromosomes) of the leptin gene, which causes an absence of leptin and leads to a constant desire for food and severe obesity. Certain subjects may have a leptin signaling deficiency due to heterozygous mutations (Ob/+) at the leptin gene, which, although not as severe, may still lead to an increased desire for food and increased risk of obesity because of reduced leptin levels. Such subjects may be considered to have a leptin signaling deficiency.

Alternatively, certain obese subjects have unusually high circulating concentrations of leptin. These subjects are often considered resistant to the effects of leptin. It is believed that the high sustained concentrations of leptin from the enlarged adipose stores may result in leptin desensitization. Regardless of the specific mechanism, and likewise to the absence or reduction of leptin, the body doesn't adequately receive the feeling of satiety subsequent to eating. Such subjects may also be considered to have a leptin signaling deficiency.

It has also been established that the brains of obese men and women exhibit less activation in the left dorsolateral prefrontal cortex (LDLPFC) in response to a meal than is seen in lean men or women, indicating an association between this altered neuronal response and the pathophysiology of obesity. Decreased LDLPFC in response to a meal activity may be measured according to techniques known in the art and described herein (see, e.g., Le et al., Am J Clin Nutr 86:573-579, 2007; and Le et al., Am J Clin Nutr 84:724-31, 2006, each of which is herein incorporated by reference in its entirety).

A “hedonic” response response to a food portion relates generally to the perceived pleasantness of that food portion by a given user. It is believed that such hedonic responses may modulate appetite control, such as by influencing the choice of foods. For instance, studies have established a positive relationship between the rated pleasantness of fatty foods and the adiposity of the subjects (see, e.g., Mella and Sacchetti, Am J Clin Nutr 53:908-15, 1991). Hedonic responses to food have also been associated with food addiction, analogous to the manner in which subjects become addicted to drugs (see, e.g., Appetite 42:131-8, 2004).

Suitable users (e.g., patients or subjects) include laboratory animals (e.g., mice, rats, rabbits, guinea pigs, etc.), farm animals, and domestic animals or pets (e.g., cats, dogs, etc.). Non-human primates and more preferably human users are included in certain preferred embodiments contemplated herein.

As described herein, reducing food consumption relates generally to a decrease in the amount of food and/or drink that a user consumes in a given meal (i.e., breakfast, lunch, dinner), in a given day, and/or in any other relevant time frame (e.g., minutes, hours, weeks, months, years, etc.), as compared to when a user-interactive system of the invention is not employed. The comparison may be made between two different users, or the same user at different times. The reduced or decreased amount of food or drink may be measured by the weight of the food, its energy value (e.g., calories), the volume of the food, and/or any other relevant measurement known in the art or described herein. A reduced or decreased amount is thus typically a statistically significant reduction or decrease, and may include a decrease that is 0.99, 0.9, 0.8, 0.7, 0.6, 0.5, 0.4, 0.3, 0.2, 0.1, 0.05, 0.04, 0.03, 0.02, 0.01, or less times an original amount or a control amount (including all integers and decimal points in between and below 1).

Certain embodiments related to altering a behavioral, neurological, psychological, or physiological response, which relates generally to increasing or decreasing such a response, as described herein, typically in a statistically significant amount. A behavioral, neurological, psychological, or physiological response to a food portion may include, for example, a leptin-signaling response, a left dorsolateral prefrontal cortex activity in response to food, or a hedonistic response to food as described herein., Other responses to food which may be altered include, but are not limited to, the amount of food consumed, the time interval until hunger is next felt, the subjective multimodal sensory experiences a person feels as they eat, the emotional response to eating, etc.

A user-interactive system relates generally to any system of one or more discrete elements that function together to allow the user to view or perceive and respond to his or her actions. Examples of a user-interactive system include a computer operatively coupled to a camera means, an image-processing means and a display means, wherein the compound is programmed to control one or more of image generation, image magnification, and/or image display. A user-interactive system may also be employed without a computer, such as by merely using a camera means and an image processing means, wherein one of the camera means and/or image processing means is capable of magnifying the image as perceived by the user. In certain embodiments, the camera means, the image processing means, and the imaging means may be in the same device, such as in a video camera with a built in imaging screen.

Certain preferred embodiments as disclosed herein may be practiced without having to determine food portion size, which relates generally to avoiding any requirement for the process of planning out or calculating beforehand (e.g., prior to ingestion or consumption) the amount of food to be consumed by the user in a given meal or snack, which processes in the absence of the present embodiments are typically used to limit or manage the amount of food to be consumed. Such planning can be based on the caloric value of the food portion (e.g., “I will eat a maximum of 800 calories for this meal”), the weight or mass of the food portion (e.g., “I will eat no more than one six-ounce piece of chicken and two ounces of broccoli”), standard serving sizes, such as those set forth in a pre-packaged food portion, or any other metric that may be relevant to a food portion, as known in the art or described herein. The instant methods thus do not rely on such planning, because independent of the initial food portion, the present methods independently facilitate the process by which the user's brain signals to the body that the user has reached a satiety point, thereby signaling the user to stop or reduce eating.

A camera means includes any optical device that is capable of generating or capturing an image of a real-life object, such as a bite of food. Examples of “camera means” include, but are not limited to, closed-circuit television cameras, digital cameras, mirror devices, digital video cameras, web cameras, film cameras, film video cameras, camera phones, and projection cameras. A camera means may be wireless or wired, and may be capable of capturing a still image and/or a moving image. In certain embodiments, the camera means may be mounted on the user's body, such as a head-mounted camera (e.g., helmet or head cam). A camera means may also be capable of magnifying the image. In certain embodiments, the camera means may be attached to the display means by a support member, such as a detachable and/or flexible arm.

A “bite of food” refers generally to a single “dose” or “swallow” or “mouthfull” of food, which may include by way of non-limiting example any solid or liquid food or beverage or any other foodstuff including edible semisolids, colloids, gels, suspensions, pastes, butters, emulsions, slurries, purees, liquids and the like. For instance, a bite of food or single dose of food could be an amount of food that fits in a spoon or on a fork, or that fits in a users hand, and which is typically consumed in one or more bites or mouthfuls before the user moves on to ingesting the next bite of food. A food source may include all or a portion of a serving of food, such as that portion of food found on a user's plate or bowl during a given meal or snack. A food source may also include any other container containing food from which a user could derive a bite of food, such as a bag or box of food, or a container which holds a beverage, such as a glass or coffee cup. In certain embodiments, “bite of food” should be construed to include beverages.

An “image” relates generally to an artifact, usually two-dimensional (e.g., a picture or other visual representation of information), that has a similar appearance to some real-life subject, such as a physical object or a person. Images may be two-dimensional, such as a photograph, video or other visible screen display, as well as three-dimensional, such as in certain specialized video displays. Images may be captured by optical devices, such as cameras, mirrors, lenses, telescopes, microscopes, etc., as well as by natural objects and phenomena, such as the human eye.

A volatile image is one that exists only for a short period of time, and may include a reflection of an object in a mirror, a projection of a camera obscura, or a scene displayed on a cathode ray tube. A fixed image, also called a hard copy, is typically an image that has been recorded on a material object, such as paper or textile by photography or digital processes. A still image is a single static image, as distinguished from a moving image. An image may include an optically projected image, a television image, and/or a digital image.

An image processing means includes any device that is capable of producing and/or modifying an image that has been captured by a camera means, such as by receiving the image or information on that image from the camera means, optionally magnifying that image to obtain a magnified image, and sending that image to a display means. An image processing means may be a separate element or device, or it may be physically incorporated into one or both of the camera means and/or the display means. In certain embodiments, the image processing means may be attached to at least one of the camera means and/or the display means. An image processing means may include a computer. In certain embodiments, an image processing means may include a computer processor and a computer-readable medium having executable instructions stored thereon for converting an image signal generated by the camera means into a display signal that can be displayed by the display means.

A display means includes any device that is capable of receiving an image from a camera means or image processing means and displaying a visual representation of the image for viewing by a user, particularly wherein that image is a magnified image of a bite of food. Examples of display means include computer screens or monitors, cell phone screens, flat-screen televisions, cathode ray tube (CRT) displays, light projection screens, liquid crystal display (LCD) screens, plasma screens, and light-emitting diode (LED) screens, among others known in the art. In certain embodiments, the display means may be mounted on the user's body, such as by utilizing a head-mounted visual display apparatus.

A magnified image or “magnifying” an image, such as an image of a bite of food, relates to an increase in the perceived size of that image from the perspective of the viewer, or user, as compared to an unmagnified, or reference image or object (e.g., a real-life object). An increase in size is typically a statistically significant increase, and may include an increase in perceived size that is at least 1.1, 1.2, 1.3, 1.4, 2, 3, 4, 5, 6, 7, 8, 9, 10, 11, 12, 13, 14, 15, 16, 17, 18, 19, 20, 25, 30 or more times (including all integers and decimal points in between and above 1), e.g., 1.5, 1.6, 1.7. 1.8, 5.5, 5.6, etc.) the size of the reference or unmagnified (i.e., real-life) object or image, such as a bite of food.

A “minified image” or “minifying an image” relates to a decrease in the perceived size of that image from the perspective of the viewer, or user, as compared to an unminified, or reference image or object. A decrease in size is typically a statistically significant decrease or reduction, and may include a decrease in perceived size that is 0.99, 0.9, 0.8, 0.7, 0.6, 0.5, 0.4, 0.3, 0.2, 0.1, 0.05, 0.04, 0.03, 0.02, 0.01, or less times (including all integers and decimal points in between and below 1) the size of the unminified object or image, such as a reference object.

A reference object references generally to one or more objects of defined size that can be used to alter (e.g., increase) the relative, perceived size of the magnified image of the bite of food, the latter being viewed by the display means, and the reference object being viewed either directly by the user or via the same or different display apparatus as the bite of food. Examples of reference objects include the hand of the user, typically unmagnified, an artificial hand, a scale model of an artificial hand, an image of a hand, and an image of a scale model of a hand.

In certain embodiments, the reference object is an article of manufacture having dimensions that correspond to a manufacturing industry standard, such as an electronic or electrical device or a component thereof that is selected from a removable battery for a portable electronic device, an alternating current (AC) wall plug, an AC wall outlet, a light bulb, a compact disc, a telephone handset, and a computer mouse, or the like.

A reference object may include a unit of currency, such as a paper bill of standard denomination or a metal coin of standard denomination, a common household item, such as a twelve-ounce beverage can, a twelve-ounce beverage bottle, a playing card, a deck of playing cards, a pair of eyeglasses, a pair of sunglasses, a ruler, and/or a scale model of any of the articles of manufacture described herein. One or more reference objects may be viewed indirectly by the display means (via the user-interface system); such as with a small-scale model of a reference object that otherwise appears of normal size upon magnification, or with a reference object that is minified by a user-interface system or other means. In certain embodiments, such references objects may be viewed directly by the user, independently of the user-interface system.

In certain embodiments, such as wherein the reference object is the user's own hand, the user may present the bite of food to the camera means using a first hand of the user, and then visually compare the altered image of the bite of food to the unaltered second hand of the user (i.e., the reference object), the second hand being contralateral to the first hand; contralateral generally meaning “on the other side.”

The recitation “present to the camera” relates to the process by which a user positions the bite of food in a manner that permits an image of that bite of food to be captured by the camera means, processed, and viewed as a magnified image on the display means. The user may “present” the bite of food to the camera for a time period sufficient to allow the user to “visually compare” the reference object to the displayed magnified image of the bite of food, or the user may “present” the bite of food to the camera merely for a time sufficient to capture an image of the bite of food.

Typically, using the herein described user-interactive system, the image-processing means of which converts an image signal collected by the camera means from the bite of food by magnifying it to produce a modified signal from which a magnified image is obtained, said magnified image then being displayed on the display means, the user “visually compares” the reference object (or image of a reference object) to the displayed magnified image of the bite of food based on the perceived size of these two objects or images, such that the magnified image of the bite of food is perceived as being much larger than it would otherwise appear if not magnified. The use of a reference object of the user-interactive system as a comparison tool facilitates and/or enhances the perception that the magnified image of the bite of food is much larger than it would otherwise appear in real-life.

The user may “visually compare” the reference object to the displayed magnified image for a “time period” immediately before ingestion of the bite of food. Such a time period may include about 1, 2, 3, 4, 5, 6, 7, 8, 9, 10, 15, 20, 35 30, 35, 40, 45, 50, 60, 70, 80, 90, 100, or more seconds (including all integers in between), or any time period that is considered sufficient to allow the brain of the user to compare the reference object to the altered image of the bite of food.

A user may repeat the steps of the instant methods one or a plurality of times to reach a satiety point. A satiety point refers to the quality or state of being fed or gratified by the food already consumed, and ideally signals the end of the meal or snack. Preferably, a user that employs a user-interactive system of the present embodiments would repeat the steps of the instant methods a fewer number of times as compared to when a user-interactive system is employed that does not provide a magnified image of the bite of food, or as compared to when the user-interactive system is not used.

A “plurality,” as used herein, means more than one, and includes 2, 3, 4, 5, 6, 7, 8, 9, 10, 15, 20, 35 30, 35, 40, 45, 50, 60, 70, 80, 90, 100, or more (including all integers in between).

In certain embodiments, a user-interactive system may also comprise a lighting means for illuminating the bite of food when it is presented to the camera means. A lighting means may include any source of light, such as a flashlight, a light bulb, a LED light, a xenon light, a headlamp, a strobe light, a lamp, or the like. This lighting means, may be controlled by the user, automated, or be a combination of automated and user controlled. In certain embodiments, a lighting means may also include manipulation of the image by the image processing means. A lighting means during image processing may include, but is not limited to, gamma correction, brightness, color tone or sharpness. Such image processing may be automated, controlled by the user, or some combination of automated and user controlled.

In certain embodiments, a user-interactive system may also comprise a protective tray, which may be used to protect any one or more of the camera means, the image processing means, and/or the display means from food or drink spillage by the user or any other nearby subject. A protective tray may be partially or fully composed of one or more see-through or transparent materials, particularly if used to protect the camera means and/or the display means.

Certain embodiments contemplate an apparatus distinct from the above-described user-interactive system having, inter alia, a camera means, an image-processing means and a display means, for use in the methods provided herein. An example of such a distinct apparatus may comprise a magnifying lens or eyeglass that magnifies an object viewed by a user by a multiplication factor referred to as “factor X.” A “factor X” may thus include a multiplication factor with X having a value of 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, or more (including all integers and decimal points in between and above 1). Such an apparatus may also include a 1/X scale model of a reference object, such as a human hand. The value “X” in a “1/X” scale model may include a value of 1.1, 1.2, 1.3, 1.4, 1.5, 1.6, 1.7, 1.8, 1.9, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 16, 17, 18, 19, 20, 25, 30, 35, 40, 45, 50, or more (including all integers and decimal points in between and above 1).

Certain specific embodiments may include a thin monitor that stands in the user's line of sight behind the food source (e.g., a plate containing food) with a retractable arm that provides a camera to target the bite of food, and a source of light, which extends over an upper corner of the monitor (e.g., the upper right hand corner, in the case of a user using the right hand to eat and the contralateral (e.g., left) hand as a reference object). Here, the user may place the left hand (i.e., the non-eating hand) on the table so the left hand is near the monitor and in the user's line of sight to the monitor, and eats by bringing each bite into the field of view of the video camera; the camera magnifies the bite as the user brings it towards the camera. The user compares the magnified image of the food to the reference hand, by initiating a grasping motion with the contralateral hand, directed at the magnified image of the bite of food on the display, and then consumes the bite of food. These steps are repeated until the user reaches a satiety point.

“Treatment” or “treating,” as used herein, includes any desirable effect on the symptoms or pathology of a disease or pathological condition, and may include even minimal reductions in one or more measurable markers of the disease or condition being treated (e.g., weight loss). “Treatment” does not necessarily indicate complete eradication or cure of the disease or condition, or associated symptoms thereof.

Accordingly, the methods provided herein may in certain preferred embodiments be used to treat or otherwise reduce the risk of obesity or excess weight (such as excess body fat), and/or treat or reduce the risk of any conditions associated with obesity. Examples of conditions associated with obesity or excess weight include, but are not limited to, cardiological conditions (e.g., ischemic heart disease, angina, myocardial infarction, congestive heart failure, high blood pressure, abnormal cholesterol levels, deep vein thrombosis, pulmonary edema, etc.), dermatological conditions (e.g., acanthosis nigricans, lymphedema, cellulitis, hirsutism, intertrigo, etc.), endocrinological and reproductive conditions (e.g., diabetes mellitus, polycystic ovarian syndrome, menstrual disorders, infertility, complications during pregnancy, birth defects, intrauterine fetal death), gastrointestinal conditions (e.g., gastroesophogeal reflux disease, fatty liver disease, gallstones, hernias), neurological conditions (e.g., stroke, meralgia parasthetica, migraines, carpal tunnel syndrome, dementia, idiopathic intracranial hypertension), oncological conditions (e.g., breast, ovarian, esophogeal, colorectal, liver, pancreatic, gallbladder, stomach, endometrial, cervical, prostate, and kidney cancers, as well as non-Hodgkin's lymphoma and multiple myeloma), psychiatric conditions (e.g., depression, social stigmatization, etc.), respiratory conditions (e.g., obstructive sleep apnea, obesity hypoventilation syndrome, asthma, increased complications during general anesthesia), rheumatological and orthopedic conditions (e.g., gout, poor mobility, osteoarthritis, lower back pain, etc.), and urological and nephrological conditions (e.g., erectile dysfunction, urinary incontinence, chronic renal failure, hypogonadism, etc.), among others known in the art and/or described herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for reducing food consumption by an overweight or obese user when employing a user-interactive system while eating, relative to food consumption when the user-interactive system is not employed, and without having to determine food portion size, the method comprising:
(a) providing a user-interactive system that comprises (i) a camera means for generating an image of a bite of food that is to be consumed by the user, (ii) an image-processing means for receiving from said camera means the image of the bite of food that is to be consumed by the user and magnifying the image to obtain a magnified image of the bite of food, (iii) a display means for receiving from the image-processing means and displaying the magnified image of the bite of food to obtain a displayed magnified image of the bite of food, (iv) a food source, and (v) a reference object;
(b) allowing the user to present to the camera means a bite of food that is to be ingested, under conditions that permit the user-interactive system to generate and magnify an image of the bite of food and display a magnified image of the bite of food on the display means, said bite of food comprising all or a portion of the food source;
(c) allowing the user to visually compare the reference object to the displayed magnified image of the bite of for a time period immediately before ingestion of the bite of food;
(d) allowing the user to ingest the bite of food; and
(e) permitting the user to repeat steps (b)-(d) zero, one, or a plurality of times up to a satiety point, and thereby reducing a first level of food consumption by the user when employing the user-interactive system while eating, relative to a second level of food consumption by the user when the user-interactive system is not employed.

2. A method for altering behavioral, neurological, psychological, or physiological response to a food portion by a user including an obese or overweight user or a user at risk of weight gain, when employing a user-interactive system while eating, relative to behavioral, neurological, psychological, or physiological response to a food portion when the user-interactive system is not employed, and without having to determine food portion size, the method comprising:
(a) providing a user-interactive system that comprises (i) a camera means for generating an image of a bite of food that is to be consumed by the user, (ii) an image-processing means for receiving from said camera means the image of the bite of food that is to be consumed by the user and magnifying the image to obtain a magnified image of the bite of food, (iii) a display means for receiving from the image-processing means and displaying the magnified image of the bite of food to obtain a displayed magnified image of the bite of food, (iv) a food source, and (v) a reference object;
(b) allowing the user to present to the camera means a bite of food that is to be ingested, under conditions that permit the user-interactive system to generate and magnify an image of the bite of food and display a magnified image of the bite of food on the display means, said bite of food comprising all or a portion of the food source;
(c) allowing the user to visually compare the reference object to the displayed magnified image of the bite of food for a time period immediately before ingestion of the bite of food;
(d) allowing the user to ingest the bite of food; and
(e) permitting the user to repeat steps (b)-(d) zero, one, or a plurality of times up to a satiety point,
and thereby altering a first level of behavioral, neurological, psychological, or physiological response to a food portion by the user when employing the user-interactive system while eating, relative to a second level of behavioral, neurological, psychological, or physiological response to the food portion by the user when the user-interactive system is not employed.

3. The method of either claim 1 or claim 2 wherein the reference object is a hand of the user.

4. The method of either claim 1 or claim 2 wherein the reference object is selected from the group consisting of an artificial hand, a scale model of an artificial hand, an image of a hand and an image of a scale model of a hand.

5. The method of claim 4 wherein the artificial hand is an artificial human hand and the image of a hand is an image of a human hand.

6. The method of either claim 1 or claim 2 wherein the reference object is an article of manufacture having dimensions that conform to a manufacturing industry standard.

7. The method of claim 6 wherein the article of manufacture is selected from the group consisting of:
(a) an electronic or electrical device or a component thereof that is selected from the group consisting of: a removable battery for a portable electronic device, an alternating current (AC) wall plug, an AC wall outlet, a light bulb, a compact disc, a telephone handset, a computer mouse,
(b) a unit of currency that is selected from a paper bill of standard denomination and a metal coin of standard denomination,
(c) a common household item that is selected from a twelve-ounce beverage can, a twelve-ounce beverage bottle, a playing card, a deck of playing cards, a pair of eyeglasses, a pair of sunglasses, a ruler,
(d) a scale model of any article of manufacture according to (a)-(c).

8. The method of either claim 1 or claim 2 wherein the user is selected from an overweight user, an obese user, a user having one or more genetic, familial or environmental risk factors for an obesity-linked cancer, a user having one or more genetic, familial or environmental risk factors for stroke, a user having a history of yo-yo dieting or weight cycling, and a user having a condition that is selected from Type 1 diabetes, Type 2 diabetes, impaired glucose tolerance, insulin resistance, sleep apnea, osteoarthritis, gout, high blood pressure, and abnormal blood lipid levels.

9. The method of either claim 1 or claim 2 wherein the user has (i) a leptin signaling deficiency, (ii) decreased left dorsolateral prefrontal cortex (LDLPFC) activity in response to a meal, or (iii) an unhealthy hedonic response to a food portion.

10. The method of claim 9 wherein the leptin signaling deficiency is modulated by at least one of (i) extending duration of the time period during which the reference object is visually compared to the displayed magnified image, and (ii) increasing a degree of magnification of the magnified image.

11. The method of either claim 1 or claim 2 wherein the camera means is selected from the group consisting of a closed-circuit television camera, a digital camera, a digital video camera, a web camera and a projection camera.

12. The method of either claim 1 or claim 2 wherein the camera means is attached to the display means by a support member.

13. The method of claim 11 wherein the image-processing means is attached to at least one of the camera means and the display means.

14. The method of either claim 1 or claim 2 wherein the image is selected from the group consisting of an optically projected image, a television image and a digital image.

15. The method of either claim 1 or claim 2 wherein the user-interactive system comprises a computer operatively coupled to the camera means, the image-processing means and the display means, said computer being programmed to control one or more of image generation, image magnification and image display.

16. The method of either claim 1 or claim 2 wherein the display means is selected from a computer screen, a flat-screen television, a cathode ray tube (CRT) display and a light-projection screen.

17. The method of either claim 1 or claim 2 wherein the user-interactive system comprises a lighting means for illuminating the bite of food when it is presented to the camera means.

18. The method of either claim 1 or claim 2 wherein in step (b) the user presents the bite of food to the camera using a first hand of the user, and wherein in step (c) the reference object that is visually compared is a second hand of the user, said second hand being contralateral to said first hand.

19. The method of either claim 1 or claim 2 wherein the image-processing means comprises a computer processor and a computer-readable medium having executable instructions stored thereon for converting an image signal generated by the camera means into a display signal that can be displayed by the display means.

20. The method of either claim 1 or claim 2 wherein the user-interactive system includes a protective covering such as a tray that protects one or more of the camera means, the image-processing means and the display means from food spillage.

21. The method of either claim 1 or claim 2 wherein the display means comprises a head-mounted visual display apparatus, and wherein in step (c) the user is allowed (i) to visually compare the reference object to one or a plurality of displayed magnified, minified, or both magnified and minified images of the bite of food, said plurality comprising images having different magnification factors, or (ii) to visually compare one or a plurality of magnified, minified or both magnified and minified images of the reference object to one or a plurality of displayed magnified, minified, or both magnified and minified images of the bite of food, said plurality of images of the bite of food and said plurality of images of the reference object each comprising images having different magnification factors.
